(19) United States
(12) Patent Application Publication
(10) Pub. No.: US 2008/0287895 A1
(43) Pub. Date: Nov. 20, 2008

(54) Method and device for applying a condom

(76) Inventor: Francis Ouellet, Pierrefonds (CA)

Correspondence Address: Louis Tessier, C.P. 54029, Mount-Royal, QC, H3P 3H4, CA

(21) Appl. No.: 12/153,420

(22) Filed: May 19, 2008

Related U.S. Application Data
(60) Provisional application No. 60/924,484, filed on May 17, 2007.

(51) Int. Cl. A61F 5/453

(52) U.S. Cl. 604/349

(57) ABSTRACT

A method for applying a condom onto a penis, the method comprising: applying the condom onto an expanding device; inserting at least a portion of the expanding device in a receiving component chamber defined by a condom receiving component through a receiving component proximal aperture leading into the receiving component chamber with the condom applied thereonto; expanding the condom using the expanding device with the condom applied thereonto; sealing the condom against the chamber peripheral surface delimiting the receiving component chamber so as to prevent ambient air from entering between the chamber peripheral surface and at least a section of the condom by creating a seal between the condom and the chamber peripheral surface; retracting the expanding device and removing the expanding device deprived of the condom from the receiving component chamber; inserting the penis of an intended user in the receiving component chamber with the condom sealed against the chamber peripheral surface; breaking the seal between the condom and the chamber peripheral surface to allow the condom to retract onto the penis; and transferring the condom from the condom receiving component to the penis.

This Application claims priority from U.S. provisional patent application Ser. No. 60/924,484 filed May 17, 2007.

### FIELD OF THE INVENTION

The present invention relates to the general field of medical devices, and is particularly concerned with a method and a device for applying a condom.

### BACKGROUND OF THE INVENTION

Urinary condoms are devices used, for example, by male patients who suffer from low bladder control or who have reduced mobility and, therefore, reduced capability of urinating in a conventional manner without assistance. A urinary condom is a device similar to a standard condom from which the tip has been removed. Instead of the typically reservoir-shaped tip of the condom, a tube extends axially from the remainder of the condom. The tube is usable to collect urine and is connectable to a urine collecting bladder.

Urinary condoms are typically relatively difficult to properly apply on the penis of the intended user. Indeed, such condoms are usually applied when the penis is in a flaccid state. Since condoms are typically conceived for being applied on an erect penis, they are relatively difficult to stretch and position properly if the penis is not in this state. Therefore, intended users of urinary condoms often either have to take a relatively large amount of time to properly position the condom or, in the case of intended users having reduced motor control, need to have someone else to help them in applying the condom.

In addition, people suffering from some conditions have relatively non-rigid penises even when in erection. Therefore, such users may also have some difficulty when using regular condoms on the penis before intercourse.

Many devices exist which allow to position either a urinary or a regular condom on the penis of an intended user. Typically, the condom is positioned inside a tube having an inside diameter larger than the diameter of a typical penis and expanded so as to abut against the inside surface of the tube. Afterwards, the penis is positioned inside the condom and the condom is released from the tube so as to relatively tightly fit around the penis. However, many of these devices use either the withdrawal of air from a space between the condom and the tube or air pushed inside the condom so as to pressurize the condom onto the inner wall of the tube. This need to use air is relatively impractical, as many intended users would have difficulty to exert the proper pressures using their lungs if the air was provided by blowing. Also, some devices alleviate this problem by using a source of vacuum or pressurized air, but this greatly increases the cost and complexity of such devices.

Against this background, there exists a need in the industry to provide improved methods and devices for applying a condom. An object of the present invention is therefore to provide such methods and devices.

### SUMMARY OF THE INVENTION

In a first broad aspect, the invention provides a condom applicator usable for applying a condom onto a penis, the condom being substantially resiliently deformable between a condom retracted configuration and a condom expanded configuration, the condom applicator comprising: an expanding device for applying the condom thereonto and subsequently expanding the condom, the expanding device having a substantially elongated configuration, the expanding device being configurable between an expanding device retracted configuration and an expanding device expanded configuration, the expanding device being substantially radially larger in the expanding device expanded configuration than in the expanding device retracted configuration; and a condom receiving component, the condom receiving component defining a receiving component chamber delimited by a chamber peripheral surface, the condom receiving component defining a receiving component proximal aperture leading into the receiving component chamber; the condom receiving component including a reversible seal creator for creating a seal between at least a portion of the condom and the condom receiving component for sealing the at least a portion of the condom against the chamber peripheral surface with the condom in the condom expanded configuration, wherein when the least a portion of the condom is sealed against the chamber peripheral surface, the condom is maintainable in the condom expanded configuration in the receiving component chamber and when the seal is broken, the condom is allowed to retract away from the chamber peripheral surface towards the condom retracted configuration.

In typical use, the condom is applied onto the expanding device with the expanding device in the expanding device retracted configuration. Then, the expanding device is inserted into the receiving component chamber and expanded into the expanding device expanded configuration. The condom is sealed against the chamber peripheral surface of the condom receiving device, which allows configuring the expanding device to the expanding device retracted configuration and removing the expanding device from the condom receiving component. The penis of the intended user is inserted in the condom receiving component and the condom is released from the condom receiving component so as to become positioned on the penis of the intended user.

Advantageously, the proposed condom applicator is relatively easy to use and relatively cost effective to manufacture. Use of the condom using the proposed condom applicator is facilitated and involves a small number of relatively quick and ergonomic steps.

In addition, the expanding device is typically manually operated, which reduces manufacturing costs and facilitates operation of the expanding device.

The proposed condom applicator is usable both with regular condoms and with urinary condoms, depending on the embodiment of the invention. Also, the proposed condom applicator is usable with many different types of condoms and is re-usable for use after one condom has been applied.

The proposed condom applicator is relatively easily washable after use and, in some embodiments of the invention, may be sterilized so as to be usable on many different patients. Also, the condom applicator typically does not contact the intended user, except for the hands used to manipulate the condom applicator, which further reduces the risks of cross-contamination when the condom applicator is used by many intended users.

In another broad aspect, the invention provides a method for applying a condom onto a penis using a condom receiving component and an expanding device, the condom receiving component defining a receiving component chamber and a receiving component proximal aperture leading into the receiving component chamber, the receiving component chamber being delimited by a chamber peripheral surface, the expanding device having a substantially elongated configuration, the expanding device being configurable between a retracted configuration and an expanded configuration, the expanding device being substantially radially larger in the expanded configuration than in the retracted configuration, the method comprising:
applying the condom onto the expanding device;
inserting at least a portion of the expanding device in the receiving component chamber through the receiving component proximal aperture with the condom applied thereonto;
expanding the condom by expanding the expanding device to the expanded configuration with the condom applied thereonto;
sealing the condom against the chamber peripheral surface so as to prevent ambient air from entering between the chamber peripheral surface and at least a section of the condom by creating a seal between the condom and the chamber peripheral surface;
retracting the expanding device to the retracted configuration and removing the expanding device deprived of the condom from the receiving component chamber;
inserting the penis in the receiving component chamber with the condom sealed against the chamber peripheral surface;
breaking the seal between the condom and the chamber peripheral surface to allow the condom to retract onto the penis; and
transferring the condom from the condom receiving component to the penis.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawings:

FIG. 1, in a side elevation view, illustrates a condom applicator in accordance with an embodiment of the present invention, the condom applicator including a condom receiving component defining a receiving component chamber and an expanding device for expanding the condom;

FIG. 2, in a perspective exploded view, illustrates the expanding device shown in FIG. 1;

FIG. 3, in a side elevation view, illustrates the expanding device shown in FIG. 2 in an expanding device retracted configuration;

FIG. 4, in a side elevation view, illustrates the expanding device shown in FIGS. 2 and 3 in an expanding device expanded configuration;

FIG. 5, in a side partial cross-sectional view, illustrates the expanding device shown in FIGS. 2 to 4 in the expanding device retracted configuration;

FIG. 6, in a side partial cross-sectional view, illustrates the expanding device shown in FIGS. 2 to 5 in the expanding device expanded configuration;

FIG. 7, in a side cross-sectional view with portions removed, illustrates the expanding device shown in FIGS. 2 to 6 in the expanding device expanded configuration expanding a condom onto the chamber peripheral surface of the condom receiving component shown in FIG. 1;

FIG. 8, in a front cross-sectional view, illustrates the expanding device shown in FIGS. 2 to 7 in the expanding device retracted configuration;

FIG. 9, in a front cross-sectional view, illustrates the expanding device shown in FIGS. 2 to 8 in the expanding device expanded configuration;

FIG. 10, in a side elevation view, illustrates a step in the use of the condom applicator shown in FIG. 1;

FIG. 11, in a side elevation view, illustrates another step in the use of the condom applicator shown in FIGS. 1 and 10;

FIG. 12, in a side elevation view, illustrates yet another step in the use of the condom applicator shown in FIGS. 1, 10, and 11;

FIG. 13, in a side cross-sectional view, illustrates yet another step in the use of the condom applicator shown in FIGS. 1 and 10 to 12;

FIG. 14, in a cross-sectional view, illustrates yet another step in the use of the condom application shown in FIGS. 1 and 10 to 13;

FIG. 15, in a side elevation view, illustrates a condom after it has been applied to the penis of an intended user using the condom applicator shown in FIGS. 1 and 10 to 14;

FIG. 16, in a partial side elevation view including a partial cross-section, illustrates a condom receiving component in accordance with an alternative embodiment of the invention;

FIG. 17, in a side cross-sectional view, illustrates a condom receiving component in accordance with another alternative embodiment of the invention, the condom receiving component being shown with a condom sealed thereto;

FIG. 18, in a side cross-sectional view, illustrates a condom receiving component shown in FIG. 17, the condom receiving component being shown with a condom unsealed therefrom; and

FIG. 19, in a side cross-sectional view, illustrates a condom receiving component in accordance with yet another alternative embodiment of the invention.

### DETAILED DESCRIPTION

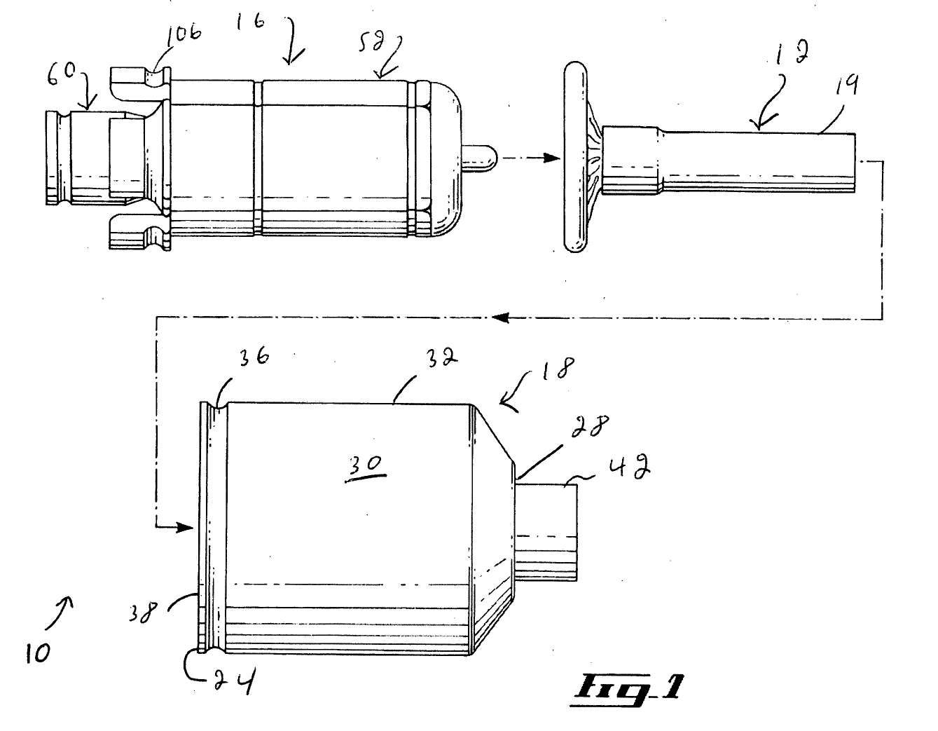

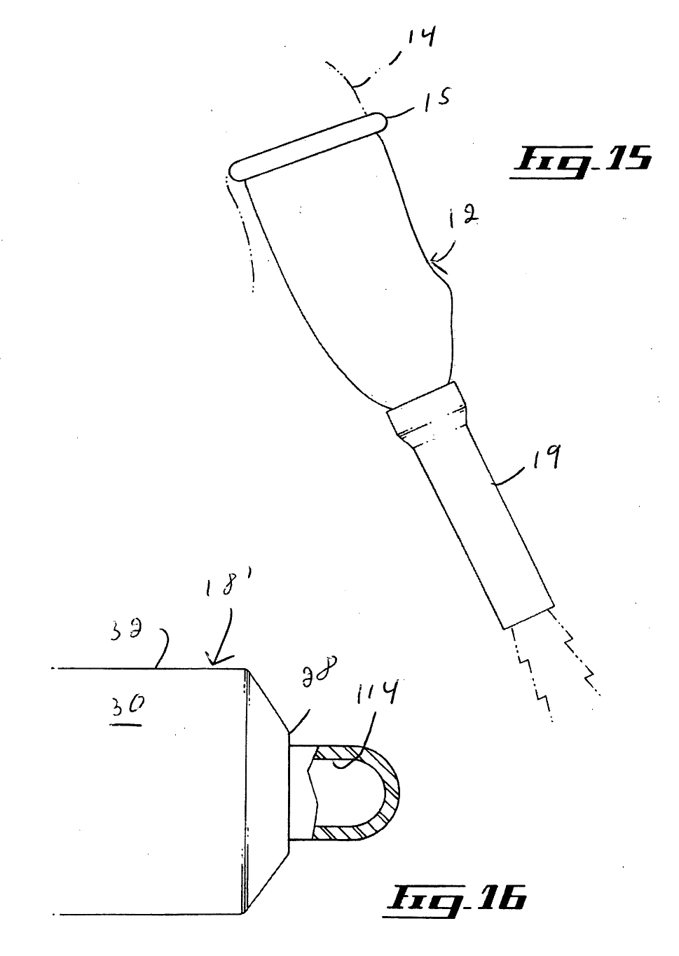

FIG. 1 illustrates a condom applicator 10 usable for applying a condom 12 onto the penis 14 of an intended user, the penis 14 being shown in FIG. 15. The condom 12 includes a conventional condom rim 15 and is substantially resiliently deformable between a condom retracted configuration shown, for example in FIG. 14, and a condom extended configuration shown, for example, in FIG. 13. In some embodiments of the invention, the condom 12 is a urinary condom 12 including a substantially axially extending condom end tube 19.

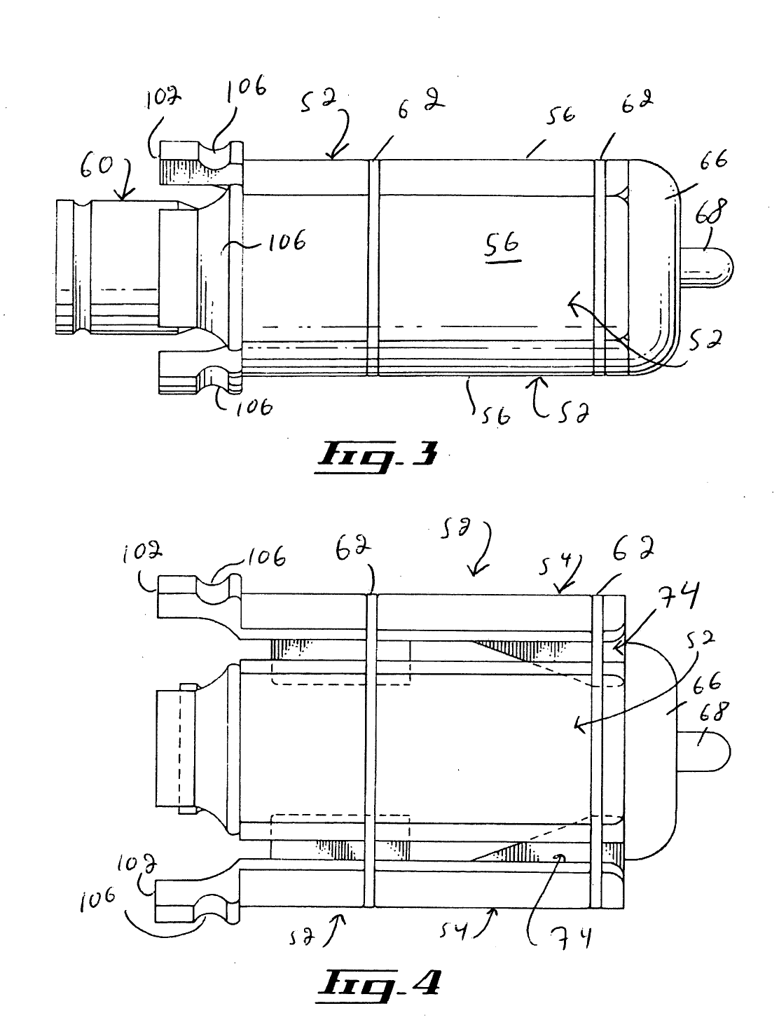

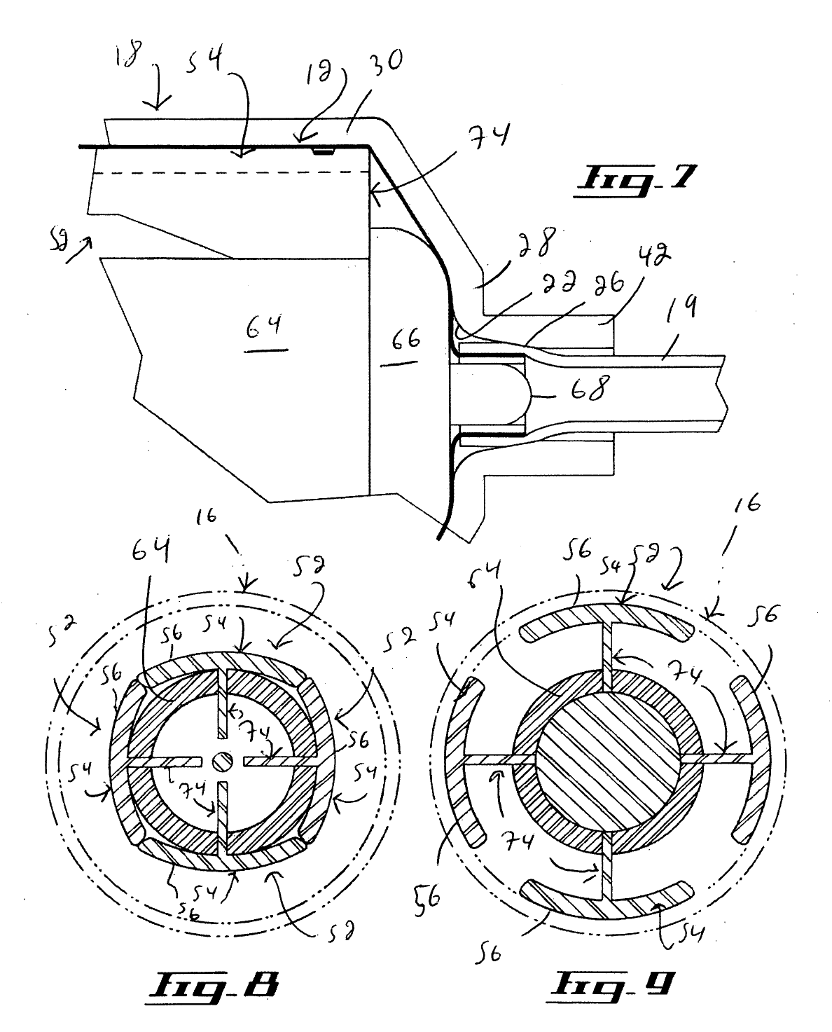

Referring to FIG. 1, the condom applicator 10 includes an expanding device 16 for applying the condom 12 thereon and subsequently expanding the condom 12 from the condom retracted configuration to the condom expanded configuration. The expanding device 16 has a substantially elongated configuration and is configurable between an expanding device retracted configuration seen, for example, in FIGS. 3, 5 and 8, and an expanding device expanded configuration shown, for example, in FIGS. 4, 6 and 9. The expanding device 16 is substantially radially larger in the expanding device expanded configuration than in the expanding device retracted configuration.

Referring to FIG. 1, the condom applicator 10 also includes a condom receiving component 18. As seen in FIGS. 13 and 14, the condom receiving component 18 defines a receiving component chamber 20 delimited by a chamber peripheral surface 22. The condom receiving component 18 defines a receiving component proximal aperture 24 leading into the receiving component chamber 20.

The condom receiving component 18 includes a reversible seal creator for creating a seal 26 as seen, for example, in FIG. 13, between at least a portion of the condom 12 and the condom receiving component 18 for sealing the at least a portion of the condom 12 against the chamber peripheral surface 22 with the condom 12 in the condom expanded configuration. When the portion of the condom 12 is sealed against the chamber peripheral surface 22, the condom 12 is maintainable in the condom expanded configuration in the receiving component chamber 20. When the seal 26 is broken as seen, for example, in FIG. 14, the condom 12 is allowed to retract away from the chamber peripheral surface 22 toward the condom retracted configuration.

As seen, for example, in FIG. 14, the condom receiving component 18 defines a receiving component distal wall 28 substantially opposed to the receiving component proximal aperture 24 and a receiving component peripheral wall 30 extending therefrom. The receiving component peripheral wall 30 defines a peripheral wall outer surface 32 and a peripheral wall inner surface 34. The peripheral wall inner surface 34 is part of the chamber peripheral surface 22.

The reversible seal creator includes a substantially circumferential receiving component groove 36 extending substantially radilally inwardly into the peripheral wall outer surface 32 substantially adjacent the receiving component proximal aperture 24. The receiving component proximal aperture 24 is delimited by a proximal aperture rim 38.

Referring to FIGS. 13 and 14, in some embodiments of the invention, the condom receiving component 18 also defines a receiving component distal aperture 40 located substantially opposed to the receiving component proximal aperture 24 and leading into the receiving component chamber 20. For example, the receiving component distal aperture 40 extends through the receiving component distal wall 28. In some embodiments of the invention, as seen for the condom applicator 10 shown in FIGS. 1 to 16, the condom receiving component 18 defines a receiving component distal end tube 42 extending substantially away from the receiving component proximal aperture 24 from the receiving component distal wall 28. However, as described in further details hereinbelow, in alternative embodiments of the invention, the receiving component distal aperture 40 is omitted from an alternative condom applicator.

Still referring to FIG. 14, the receiving component chamber 20 defines a chamber distal end section 46 substantially opposed to the receiving component proximal aperture 24. The chamber distal end section 46 tapers in a direction leading substantially away from the receiving component proximal aperture 24. For example, the chamber distal end section 46 includes a portion having a substantially frusto-conical configuration and helps in centering the condom 12 and the expanding device 16 in the receiving component chamber 20. However, any other suitable shape for the chamber distal end section 46 is within the scope of the present invention.

The receiving component chamber 20 also typically defines a chamber main section 48 extending substantially away from the chamber distal end section 46 towards the receiving component proximal aperture 24. Typically, the chamber main section 48 has a substantially cylindrical configuration.

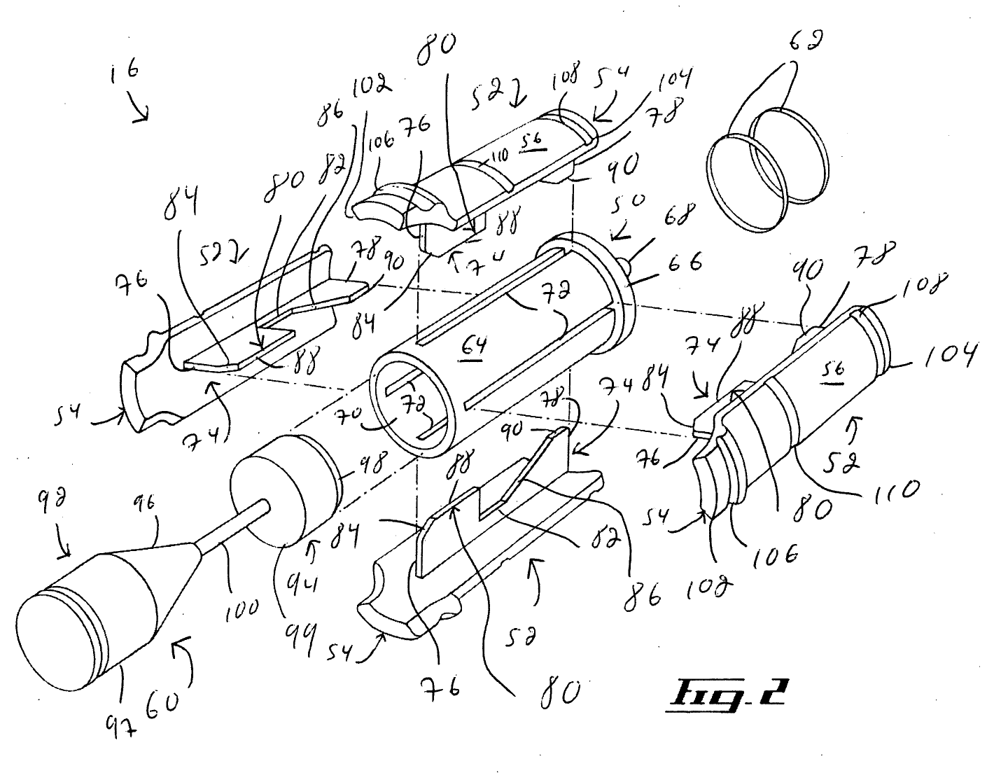

Referring to FIG. 2, the expanding device 16 includes an expanding device body 50. The expanding device body 50 typically has a substantially elongated configuration. At least one and, typically, a set of wings 52 are mounted to the expanding device body 50. Each of the wings 52 includes a condom abutment portion 54 defining a radially outermost condom abutment surface 56, which is typically substantially convex. The condom abutment surface 56 is usable for abutting a condom thereagainst when the condom 12 is applied onto the expanding device body 50. The wings 52 are mounted to the body so as to be substantially radially movable relative to the expanding device body 50 between a wing retracted position, seen for example in FIGS. 3, 5 and 8, and a wing expanded position, seen for example in FIGS. 4, 6 and 9. The condom abutment surface 56 is further away from the expanding device body 50 in the wing expanded position than in the wing retracted position.

The expanding device 16 also includes an actuator 60 for selectively moving the wing 52 between the wing retracted and expanded positions. In addition, in some embodiments of the invention, the expanding device 16 includes a pair of biasing elements 62 operatively coupled to the wings 52 for biasing the wings 52 toward the expanding device body 50.

Referring to FIG. 2, the expanding device body 50 defines a body peripheral wall 64, which typically has a substantially cylindrical configuration. A body end wall 66 extends substantially perpendicularly to the body peripheral wall 64 at an end thereof. In some embodiments of the invention, a body end protrusion 68 extends substantially longitudinally from the body end wall 66. For example, the body end protrusion 68 has a substantially rounded pointed configuration and is usable for supporting the condom end tube 19 when the condom 12 is applied onto the expanding device 16. The expanding device body 50 also defines a generally longitudinally extending body channel 70 extending from the proximal end thereof. Slots 72 extend substantially longitudinally along the body peripheral wall 64. Typically, the slots 72 extend substantially radially through the body peripheral wall 64 and lead into the body channel 70.

The wings 52 are slidably mounted to the slots 72 for radial movement relatively thereto. In addition to the condom abutment portion 54, each of the wings 52 includes an abutment portion-to-actuator link 74. Typically, the condom abutment portion 54 has a shape substantially similar to an arc segment of a cylindrical shell. The abutment portion-to-actuator link 74 is substantially plate-shaped and extends substantially perpendicularly to the condom abutment portion 54. The condom abutment surface 56 is located substantially opposed to the abutment portion-to-actuator link 74.

In some embodiments of the invention, as seen in FIG. 3, the body end wall 66 has a radius such that the edge of the body end wall 66 is substantially adjacent the condom abutment surface 56 when the expanding device 16 is in the expanding device retracted configuration, so as to facilitate the application of the condom 12 onto the expanding device 16.

Returning to FIG. 2, the abutment portion-to-actuator link 74 defines a proximal link end edge 76 and a distal link end edge 78, the link distal end edge 78 being closer to the body end wall 66 than the proximal link end edge 76. The abutment portion-to-actuator link 58 also defines a link radially innermost edge 80 located substantially opposed to the condom abutment portion 54 and extending between the proximal and distal link end edges 76 and 78.

The proximal and distal link end edges 76 and 78 extend substantially radially and are distanced by a distance substantially similar to the length of the slots 72. This allows guiding the wings 52 in a substantially radial movement through the slots 72.

The link radially innermost edge 80 defines a link recess 82 at a location intermediate the proximal and distal link end edges 76 and 78. The link radially innermost edge 80 also defines two link ramps 84 and 86. The first link ramp 84 extends from the proximal link end edge 76 and is angled so as to converge radially inwardly in a direction leading toward the distal link end edge 78. The other link ramp 86 extends from within the link recess 82 and is also angled so as to converge radially inwardly in a direction leading toward the distal link end edge 78. Each of the link ramps 84, 86 ends at a respective link longitudinal edge portions 88 and 90 that extend substantially longitudinally. The first link longitudinal edge portion 88 ends at the link recess 82, while the other link longitudinal edge portion 90 ends at the distal link end edge 78.

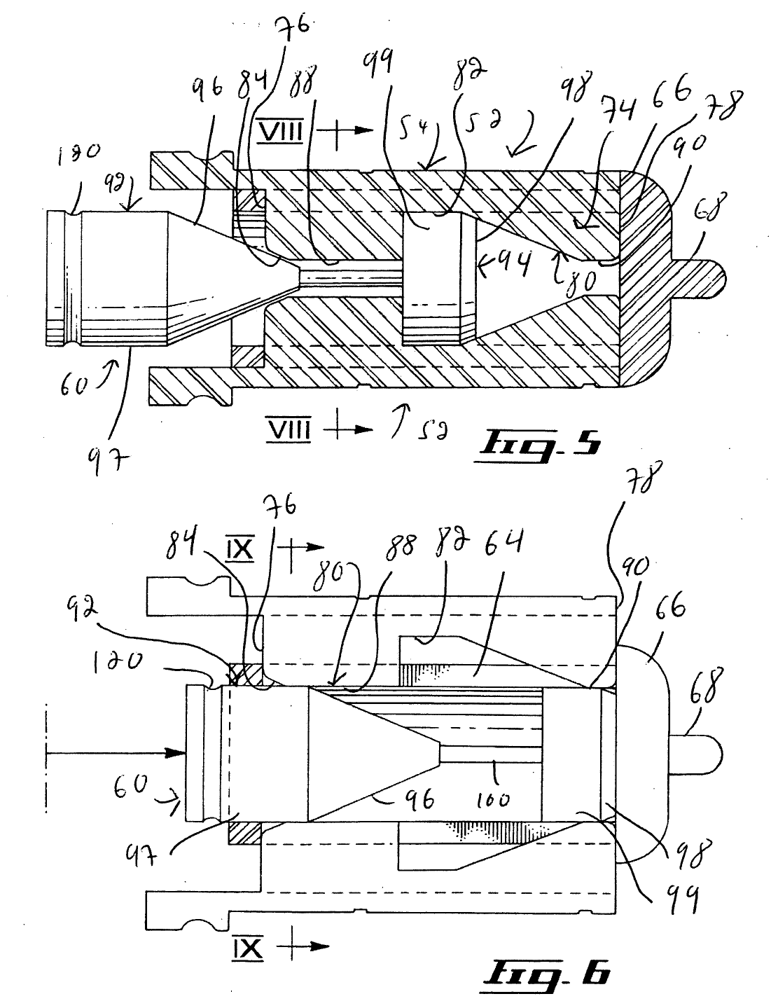

The actuator 60 is movable between an actuator engaged position, seen for example in FIGS. 4 and 6 and an actuator retracted position, seen for example in FIGS. 3 and 5. In the actuator retracted position, the wings 52 are in the wing retracted position. When the actuator 60 is in the actuator engaged position, the wings 52 are in the wing expanded position.

The actuator 60 includes actuator first and second actuating components 92 and 94. The actuator first and second actuating components 92 and 94 are substantially longitudinally spaced apart and linked to each other through an actuating component link 100. Each of the actuator first and second actuating components 92 and 94 includes a respective actuating component wing moving portion 96 and 98 tapering towards the body end wall 66 and a respective actuating component wing maintaining portion 97 and 99 extending from a respective actuating component wing moving portion 96 and 98 substantially away from the body end wall 66.

Typically, the actuating component wing moving portions 96 and 98 are substantially frusto-conical and are angled so as to engage the link ramps 84 and 86 in a substantially parallel relationship relatively thereto. Also, typically, the actuating component wing maintaining portions 97 and 99 are substantially cylindrical. When assembled, the actuator 60 is located, at least in part, inside the body channel 70. Positioning of the actuator 60 relatively to the wings 52 in the wing retracted and expanded configurations is described hereinbelow.

The reader skilled in the art will readily appreciate that while the actuator 60 includes two actuating components 92 and 94, each provided for engaging a respective link ramp 84 and 86, alternative embodiments of the invention include any other number of actuating components and ramps. Also, while a specific manner of radially moving the wings 52 is disclosed herein, the wings 52 may be moved substantially radially in any other suitable manner.

In some embodiments of the invention, the wings 52 define a wing proximal end section 102 and a wing distal end section 104. A wing groove 106 extending substantially radially inwardly is defined in the wing proximal end section 102. The wing proximal end section 102 is typically radially prominent relatively to the remaining portions of the wings 52. In some embodiments of the invention, a wing groove 108 extends radially inwardly into the wing 52 substantially adjacent the wing distal end section 104 while another wing groove 110 extends radially inwardly into the wings 52 substantially intermediate the wing proximal and distal end sections 102 and 104.

When the wings 52 are mounted to the expanding device body 50 in an operative configurations, biasing elements 62 in the form of elastic bands are positioned inside the wing grooves 108 and 110 so as to bias the expanding device 16 toward the expanding device retracted configuration.

In some embodiments of the invention, as shown in the drawings, the expanding device 16 includes four wings 52 and, therefore, four slots 44, circumferentially spaced apart about equally from each other. However, it is within the scope of the present invention to have an expanding device 16 having any other suitable shape, including any other number of wings 52. Typically, the wings 52 are configured in a manner such that their lateral edges are substantially adjacent to the lateral edge of adjacent wings when the wings 52 are in the wing retracted position.

Generally speaking, the condom applicator 10 is usable for performing a method for applying the condom 12 onto the penis 14. However, a similar method is usable using any other suitable alternative condom applicator.

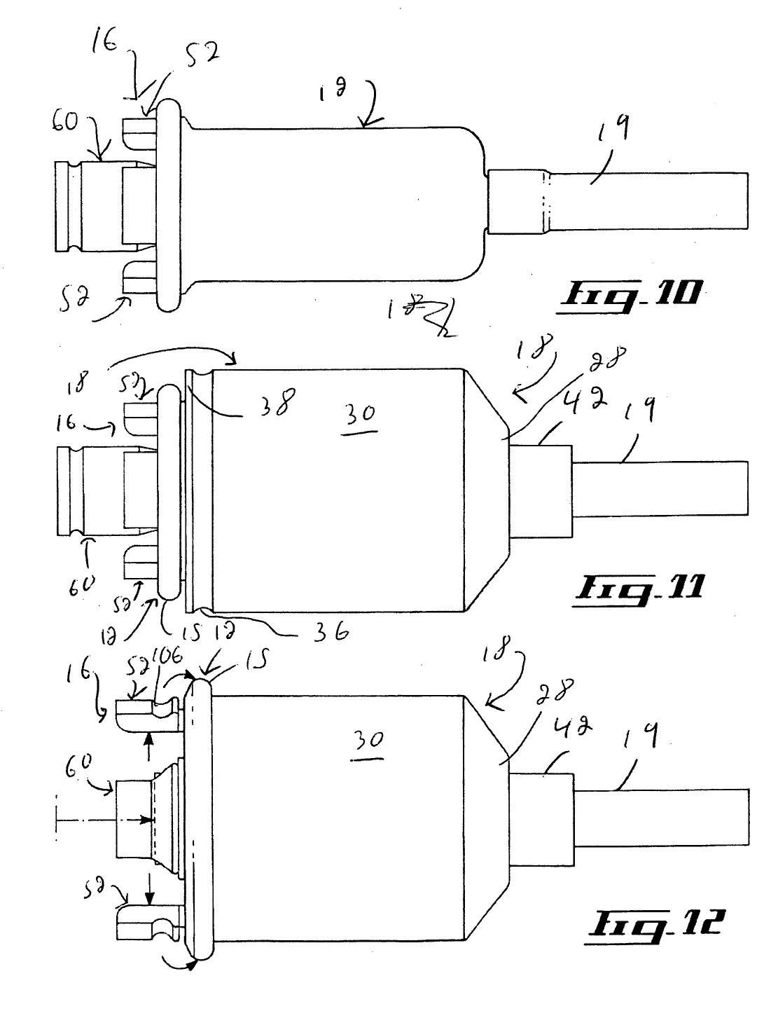

The method is illustrated with reference to FIGS. 10 to 15. The method includes first applying the condom 12 onto the expanding device 16, as shown in FIG. 10. Then, as seen in FIG. 11, at least a portion of the expanding device 16 is inserted in the receiving component chamber 20 through the receiving component proximal aperture 24 with the condom 12 applied thereonto.

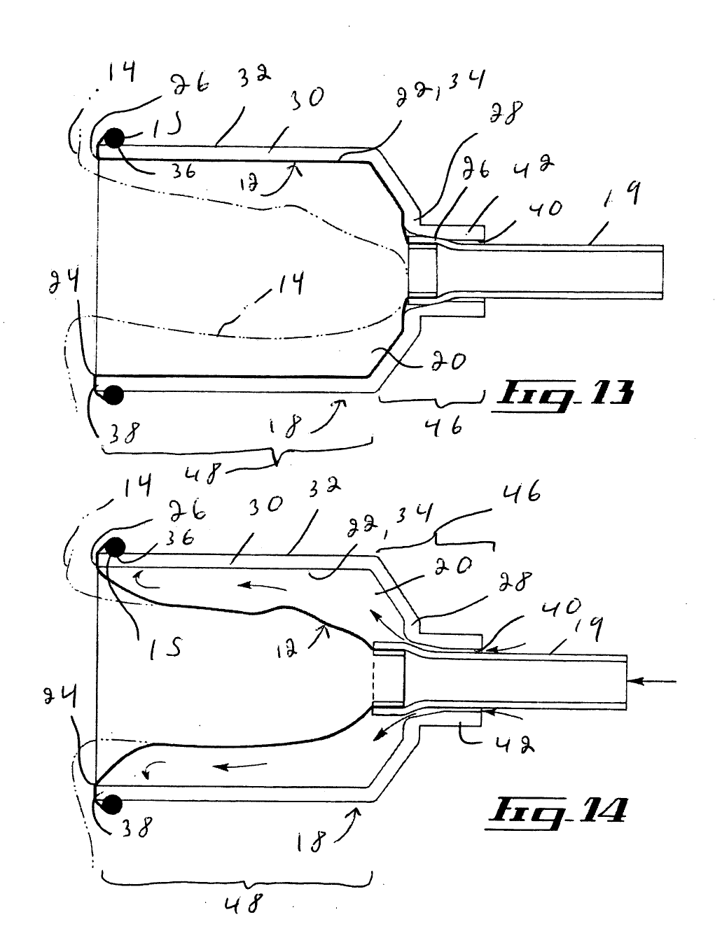

Afterwards, as seen in FIG. 12, the condom 12 is expanded by expanding the expanding device 16 to the expanding device expanded configuration with the condom 12 applied thereonto. Afterwards, as seen with reference to FIGS. 12 and 13, the condom 12 is sealed against the chamber peripheral surface 22 so as to prevent ambient air from entering between the chamber peripheral surface 22 and at least a section of the condom 12 by creating a seal 26 between the condom 12 and the chamber peripheral surface 22. Subsequently, the expanding device 16 is retracted into the expanding device retracted configuration and removed from the receiving component chamber 20 deprived of the condom 12. This is made possible by the fact that at this stage, the condom 12 has been transferred to the condom receiving component 18 and is maintained thereonto because of the seal created between the condom 12 and the chamber peripheral surface 22.

Afterwards, as seen in FIG. 13, the penis 14 is inserted in the receiving component chamber 20 with the condom 12 sealed against the chamber peripheral surface 22. Subsequently, the method includes breaking the seal between the condom 12 and the chamber peripheral surface 22 to allow the condom to retract onto the penis 14, as seen in FIG. 14, and transferring the condom 12 from the condom receiving component 18 to the penis 14, as seen in FIG. 15.

Typically, inserting the expanding device 16 in the receiving component chamber 20 is performed with the expanding device 16 in the expanding device retracted configuration and expanding the condom 12 by expanding the expanding device 16 is performed after having inserted the expanding device 16 in the receiving component chamber 20. However, in alternative embodiments of the invention, this sequence of events is reversed and the expanding device 16 is inserted in the receiving component chamber 20 with the expanding device 16 in the expanding device expanded configuration.

In some embodiments of the invention, sealing the condom 12 against the chamber peripheral surface 22 creates two substantially annular seals 26 at two substantially longitudinally spaced apart locations. As seen in FIG. 13, this is created, for example, by folding the condom rim 15 over the proximal aperture rim 38 and inserting the condom rim 16 into the receiving component groove 36 for securing the condom rim 15 thereto. In these embodiments, the condom rim 15 is folded over the proximal aperture rim 38 so that the condom rim 15 is located outside of the receiving component chamber 20.

The second substantially annular seal is performed opposed to the condom rim 15 by abutting and sealing the condom 12 against the chamber peripheral surface 22. For example, when the condom 12 is a urinary condom, the condom end tube 19 is inserted through the distal end tube 42, which is typically dimensioned such that the condom end tube 19 is substantially radially inwardly deformed and, therefore, through its resilient nature, biased against the chamber peripheral surface 22 in the distal end tube 42, or at any other location substantially adjacent the receiving component distal aperture 40.

As seen in FIG. 1, the condom 12 is typically provided with a peripheral wall thereof rolled around the condom rim 15. In these embodiments, applying the condom 12 onto the expanding device 16 includes unrolling the condom 12 onto the expanding device and, more specifically, over the wings 52 so that the condom 12 is supported by the condom abutment surfaces 56 of the wings 52, typically with the condom rim 15 receiving in the wing groove 106 to better secure the condom 12 to the expanding device 16.

Expanding the condom 12 is typically performed by using the actuator 60 for moving the wings 52 from the wing retracted position to the wing expanded position, as described in further details hereinabove. Similarly, when the expanding device is retracted to the expanding device retracted configuration, the actuator 60 is also used for moving the wings 52 toward the wing retracted position. As seen in FIG. 14, breaking the seal includes typically pushing onto the condom end tube 19 toward the receiving component proximal aperture. This action will completely break the seal 26 between the condom 12 and the chamber peripheral surface 22. Afterwards, it is relatively easy to remove the condom rim 16 from the receiving component groove 36 to transfer completely the condom 12 onto the penis 14.

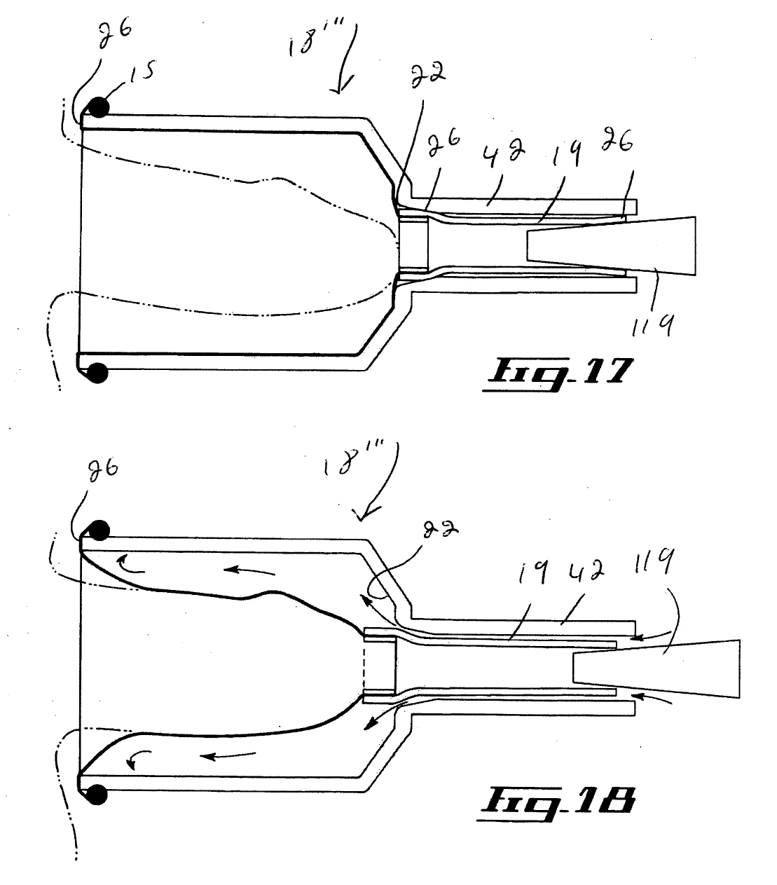

As seen in FIGS. 16 and 19, in alternative condom applicators, the condom receiving component 18′ and 18″ respectively does not include a receiving component distal aperture 40 and is instead deprived of any other aperture than the receiving component proximal aperture 24. Typically, the receiving component chamber 20 in these embodiments defines a distal recess 114 for receiving thereinto the condom end tube 19 or in embodiments in which a regular condom (not shown in the drawings) is used, a condom reservoir.

In some embodiments of the invention, for example, in the receiving component 18″, a cap 119 is used for sealing the condom end tube 19 prior to insertion into the receiving component chamber 20. The cap 119 typically engages the condom end tube 19 so as to substantially resiliently deform the condom end tube 19 to create a seal between the condom end tube 19 and the cap 119.

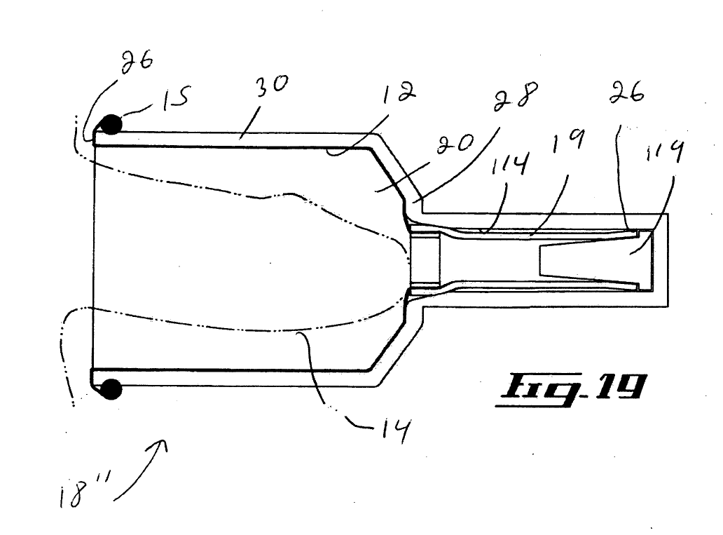

As seen in FIGS. 17 and 18, in yet other embodiments of the invention, a condom receiving component 18′″ substantially similar to the condom receiving component 18 is used with the cap 119. The cap 119 is inserted in the condom end tube 19 and biases the condom end tube 19 against the condom receiving component 18 or, more specifically, against the chamber peripheral surface 22 in the distal end tube 42. In these embodiments, breaking the seal 26 between the condom 12 and the chamber peripheral surface 22 includes removing the cap 119 from the condom end tube 19. Otherwise, the alternative condom receiving component 18′″ is usable substantially similarly to the previously described condom receiving component 18.

FIGS. 5 and 6 better illustrate the operation of the expanding device 16 between the expanding device retracted and expanded configurations. As seen in FIG. 5, in the retracted configuration, the actuator second actuating component 94 is received within the link recess 82 and the actuator first actuating component 92 is located substantially longitudinally spaced apart from the wings 52. The actuating component wing moving portions 96 and 98 engage the link ramps 84 and 86.

When the intended user exerts a longitudinal force toward the body end wall 66 onto the actuator 60, the actuating component wing moving portions 96 and 98 exert a force onto the link ramps 84 and 86 having a substantially radially outwardly directed component, which moves the wings 52 substantially radially outwardly relatively to the expanding device body 50. In the expanded configuration, as seen in FIG. 6, the actuating component wing maintaining portions 97 and 99 abut respectively against the link longitudinal edge portions 88 and 90 and therefore prevent the wings 52 from retracting toward the wing retracted position under the action of the biasing elements 62 (not shown in FIG. 6).

Pulling onto the actuator 60 away from the body end wall 66 reverses the above-described operation and allows retraction of the wings 52 towards their retracted positions. In some embodiments of the invention, the actuator 60 is provided with a substantially annular groove 120, or any other suitable grip facilitating means for improving the grip of the intended user thereonto so as to facilitate this movement.

Although the present invention has been described hereinabove by way of preferred embodiments thereof, it can be modified, without departing from the spirit and nature of the subject invention as defined in the appended claims.

What is claimed is:

1. A condom applicator usable for applying a condom onto a penis, said condom being substantially resiliently deformable between a condom retracted configuration and a condom expanded configuration, said condom applicator comprising:
an expanding device for applying said condom thereonto and subsequently expanding said condom, said expanding device having a substantially elongated configuration, said expanding device being configurable between an expanding device retracted configuration and an expanding device expanded configuration, said expanding device being substantially radially larger in said expanding device expanded configuration than in said expanding device retracted configuration; and
a condom receiving component, said condom receiving component defining a receiving component chamber delimited by a chamber peripheral surface, said condom receiving component defining a receiving component proximal aperture leading into said receiving component chamber;
said condom receiving component including a reversible seal creator for creating a seal between at least a portion of said condom and said condom receiving component for sealing said at least a portion of said condom against said chamber peripheral surface with said condom in said condom expanded configuration, wherein when said least a portion of said condom is sealed against said chamber peripheral surface, said condom is maintainable in said condom expanded configuration in said receiving component chamber and when said seal is broken, said condom is allowed to retract away from said chamber peripheral surface towards said condom retracted configuration.

2. A condom applicator as defined in claim 1, wherein said condom receiving component defines a receiving component distal aperture located substantially opposed to said receiving component proximal aperture and leading into said receiving component chamber.

3. A condom applicator as defined in claim 2, wherein said reversible seal creator includes a cap usable for selectively sealing said receiving component distal aperture.

4. A condom applicator as defined in claim 1, wherein said receiving component chamber defines a chamber distal end section substantially opposed to said receiving component proximal aperture, said chamber distal end section tapering in a direction leading substantially away from said receiving component proximal aperture.

5. A condom applicator as defined in claim 4, wherein said chamber distal end section includes a portion having a substantially frusto-conical configuration.

6. A condom applicator as defined in claim 5, wherein said receiving component chamber defines a chamber main section extending substantially away from said chamber distal end section, said chamber main section having a substantially cylindrical configuration.

7. A condom applicator as defined in claim 1, wherein said condom receiving component defines a receiving component distal wall substantially opposed to said receiving component proximal aperture and a receiving component peripheral wall extending therefrom, said receiving component distal and peripheral walls delimiting said receiving component chamber, said receiving component peripheral wall defining a peripheral wall outer surface and a peripheral wall inner surface, said reversible seal creator including a substantially circumferential receiving component groove extending substantially radially inwardly into said peripheral wall outer surface substantially adjacent said receiving component proximal aperture.

8. A condom applicator as defined in claim 1, wherein said expanding device includes
an expanding device body, said expanding device body having a substantially elongated configuration; and
a wing mounted to said expanding device body, said wing including a condom abutment portion defining a radially outermost condom abutment surface for abutting said condom thereagainst when said condom is applied onto said expanding device, said wing being mounted to said expanding device body so as to be substantially radially movable relative to said expanding device body between a wing retracted position and a wing expanded position, said condom abutment surface being further away from said expanding device body in said wing expanded position than in said wing retracted position.

9. A condom applicator as defined in claim 8, wherein said expanding device includes an actuator for selectively moving said wing between said wing retracted and expanded positions.

10. A condom applicator as defined in claim 9, wherein said expanding device includes a biasing element for biasing said wing towards said wing retracted position.

11. A method for applying a condom onto a penis using a condom receiving component and an expanding device, said condom receiving component defining a receiving component chamber and a receiving component proximal aperture leading into said receiving component chamber, said receiving component chamber being delimited by a chamber peripheral surface, said expanding device having a substantially elongated configuration, said expanding device being configurable between a retracted configuration and an expanded configuration, said expanding device being substantially radially larger in said expanded configuration than in said retracted configuration, said method comprising:
applying said condom onto said expanding device;
inserting at least a portion of said expanding device in said receiving component chamber through said receiving component proximal aperture with said condom applied thereonto;
expanding said condom by expanding said expanding device to said expanded configuration with said condom applied thereonto;
sealing said condom against said chamber peripheral surface so as to prevent ambient air from entering between said chamber peripheral surface and at least a section of said condom by creating a seal between said condom and said chamber peripheral surface;
retracting said expanding device to said retracted configuration and removing said expanding device deprived of said condom from said receiving component chamber;
inserting said penis in said receiving component chamber with said condom sealed against said chamber peripheral surface;
breaking said seal between said condom and said chamber peripheral surface to allow said condom to retract onto said penis; and
transferring said condom from said condom receiving component to said penis.

12. A method as defined in claim 11, wherein inserting said at least a portion of said expanding device in said receiving component chamber is performed with said expanding device in said retracted configuration and expanding said condom by expanding said expanding device is performed after inserting said at least a portion of said expanding device in said receiving component chamber.

13. A method as defined in claim 11, wherein sealing said condom against said chamber peripheral surface includes sealing said condom against said chamber peripheral surface along two substantially longitudinally spaced substantially annular seals.

14. A method as defined in claim 11, wherein said condom defines a condom rim and said expanding device defines an expanding device proximal rim extending peripherally to said receiving component proximal aperture, sealing said condom against said chamber peripheral surface including folding said condom rim over said expanding device proximal rim so that said condom rim is located outside of said receiving component chamber.

15. A method as defined in claim 11, wherein said condom receiving component defines a receiving component distal aperture located substantially opposed to said receiving component proximal aperture, said receiving component distal aperture leading into said receiving component chamber, sealing said condom against said chamber peripheral surface including sealing said condom against said chamber peripheral surface substantially adjacent to said receiving component distal aperture.

16. A method as defined in claim 15, wherein sealing said condom against said chamber peripheral surface includes sealing said receiving component distal aperture so as to substantially prevent air from entering between said condom and said chamber peripheral surface through said receiving component distal aperture.

17. A method as defined in claim 15, wherein said condom is a urinary condom including a substantially axially extending condom end tube, inserting said at least a portion of said expanding device in said receiving component chamber including inserting said condom end tube successively through said receiving component proximal aperture and afterwards through said receiving component distal aperture so that said condom end tube protrudes outwardly from said receiving component chamber through said receiving component distal aperture.

18. A method as defined in claim 17, said method further using a cap, wherein creating said seal includes inserting said cap in said condom end tube and biasing said condom end tube against said chamber peripheral surface with said cap.

19. A method as defined in claim 18, wherein breaking said seal includes removing said cap from said condom end tube.

20. A method as defined in claim 17, wherein breaking said seal includes moving said condom end tube towards said receiving component proximal aperture.
